(19) United States
(12) Patent Application Publication
(10) Pub. No.: US 2003/0045881 A1
(43) Pub. Date: Mar. 6, 2003

(54) Self-compressing osteosynthesis screw for surgery of small bones

(75) Inventors: Louis Samuel Barouk, Yvrac (FR); Vincent Coulange, Lyon (FR); Jean-Michel Gaume, Roanne (FR)

Correspondence Address: YOUNG & THOMPSON, 745 SOUTH 23RD STREET 2ND FLOOR, ARLINGTON, VA, 22202

(73) Assignee: DEPUY FRANCE, Saint-Priest (FR)

(21) Appl. No.: 10/227,355

(22) Filed: Aug. 26, 2002

Related U.S. Application Data
(63) Continuation of application No. 09/869,517, filed on Oct. 12, 2001.
Application No. 09/869,517 is a 371 of international application No. PCT/FR99/03321, filed on Dec. 30, 1999.

(30) Foreign Application Priority Data
Dec. 30, 1998 (FR) 98 16678

(51) Int. Cl. A61B 17/56

(52) U.S. Cl. 606/73

(57) ABSTRACT

A self-compressive and atraumatic osteosynthesis screw, includes a threaded proximal head, a body forming a threaded distal portion and, between the head and the distal part, a smooth section of short length relatively to the distal portion length, the threading thereof having a greater pitch than that of the proximal head threading, and a longitudinal channel provided from one end to the other of the screw to receive a guide pin. The screw includes a mechanism enabling it to fix small bones or small bone fragments of the skeleton after an osteotomy or a fracture, and in particular one of the five metatarsals of a human foot, including a threading diameter of the distal portion slightly greater than the diameter of the thread root of the proximal head and the diameter of the channel ranges between about 0.8 and 1.0 mm.

The present invention relates to a self-compressing and atraumatic osteosynthesis screw for surgery of small bones or of small bone fragments, in particular those of the foot.

French Patent 2,722,086 (94 08 445) describes a Barouk screw comprising a threaded proximal head, a body forming a threaded distal part and, between the head and the distal part, a smooth section whose length is short compared to the length of the distal part. The threading of the latter has a pitch greater than that of the threading of the proximal head, and a longitudinal channel is arranged from one end to the other of the body of the screw in order to receive a guide pin. The difference between the pitches of the two threadings causes a compression of the two bone fragments on one another during screwing.

This cannulated screw has a relatively great diameter because of its longitudinal channel and for this reason it can be used only for fixing fractures or for osteotomies of bones of adequate dimensions, in particular the first metatarsal in a “Scarf” osteotomy. This is because the diameter of the threaded head of this screw at the threading root is very nearly 3 mm. For smaller bones such as the other four metatarsals, when carrying out an osteotomy described by Dr Weil to correct an excess length of these metatarsals, it is necessary to use a screw of reduced dimensions such as the one described in French Patent No. 2,721,819 (94 08 373). Such a screw is continued via an endpiece which breaks off automatically by virtue of a break line formed between the head of the screw and the base of the endpiece. The reduction in the diameter of this separable screw is obtained by virtue of the fact that it does not have a longitudinal channel running through for guiding a pin.

None of these prior art screws can be used universally for osteotomy or fixation of fractures of small bones, in particular the metatarsals. This is because the screw according to U.S. Pat. No. 2,722,086 has a diameter which is too great for the four metatarsals, other than the first metatarsal, and can cause these to rupture, and for other small bones or bone fragments of the skeleton. If the bone of these four lateral metatarsals is porotic, the screw with the break-off endpiece can prove too small and its hold inadequate.

Likewise, if the first metatarsal is actually small in size, as is the case in some patients, the cannulated screw with threaded head according to the aforementioned patent risks rupturing it.

It is an object of the invention to make available an osteosynthesis screw of the abovementioned type, for surgery of small bones or small bone fragments, which can be used in place of the two known screws discussed above, not only for any one of the five metatarsals, but also for osteotomies of small bones or small bone fragments, and for fixation of fractures in applications other than surgery of the foot, for example for bones of the hand.

According to the invention, the self-compressing and atraumatic osteosynthesis screw includes means allowing it to fix the small bones or small bone fragments of the skeleton after an osteotomy or a fracture, and in particular any one of the five metatarsals of a human foot.

Thus, the screw according to the invention advantageously replaces the two screws—the cannulated one and the separable one—of the prior art set out above.

According to one embodiment of the invention, said means comprise a diameter of the threading of the distal part slightly greater than the diameter of the root of the threading of the proximal head, and the fact that the diameter of the channel is between 0.8 and 1.0 mm approximately.

This therefore results in a miniaturization of the prior art screws while at the same time maintaining the arrangement of a longitudinal channel opening out at both ends of the screw, which allows it to be guided into place using a pin.

The diameter of the root of the threading of the proximal head is approximately 2.2 to 2.7 mm, and the external diameter of the threading of the distal part is between substantially 2.5 and 3.0 mm approximately.

A screw is thus obtained whose dimensions are sufficiently reduced in relation to the screw according to French Patent 2,722,096 to allow it to be used in osteotomies or fixation of fractures of small bones such as the middle metatarsals without any risk of causing these bones to break, and at the same time the screw retains sufficient strength to resist the stresses experienced during their period of use.

Other particular features and advantages of the invention will become apparent from the following description in which reference is made to the attached drawings which show an embodiment thereof as a nonlimiting example.

FIG. 1 is a longitudinal elevation, on an enlarged scale, of an embodiment of the osteosynthesis screw according to the invention.

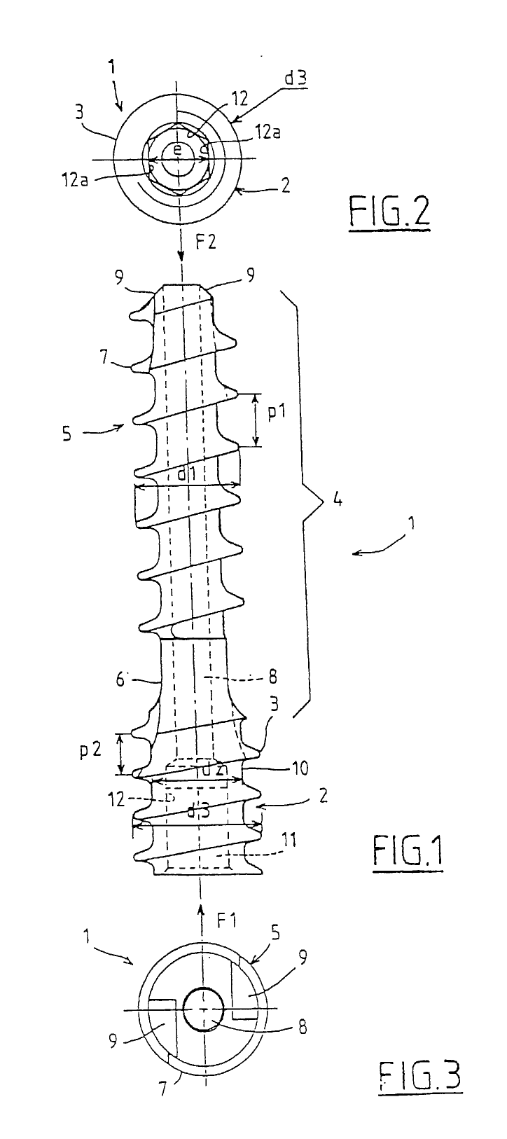

FIG. 2 is an end view of the screw from FIG. 1 in the direction of the arrow F1.

FIG. 3 is an end view of the screw from FIG. 1 in the direction of the arrow F2.

FIG. 4 is a diagrammatic plan view of the bones of a human foot, the first phalanx presenting a hallux valgus and the first metatarsal presenting a metatarsus varus, both of which can be corrected using screws according to the invention.

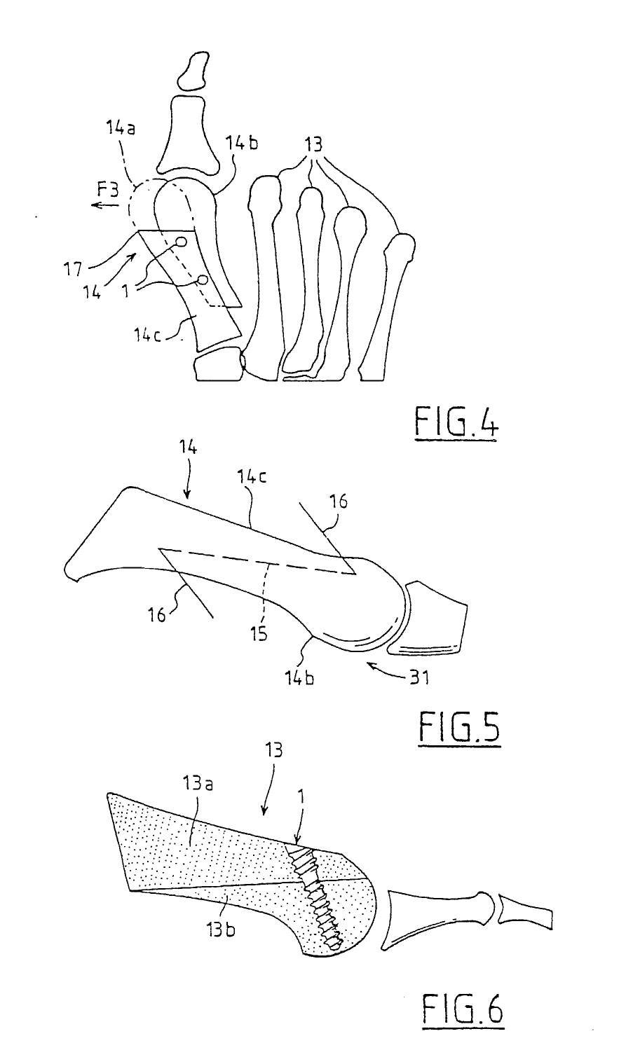

FIG. 5 is a longitudinal elevation, on an enlarged scale, of the first metatarsal from FIG. 4, showing the longitudinal and transverse lines of resection of the metatarsal bone to be treated.

FIG. 6 is a longitudinal elevation, on an enlarged scale, illustrating the use of a screw according to the invention in a Weil osteotomy of a middle metatarsal.

The osteosynthesis screw 1 for surgery of small bones or small bone fragments which is represented in FIGS. 1 to 3 comprises a proximal head 2 provided with a threading 3, and a body 4 forming a threaded distal part 5. Formed between the head 2 and the distal part 5 there is a smooth section 6 whose length is short compared to that of the distal part 5.

The threading 7 of the latter has a pitch p1 which is greater than the pitch p2 of the threading 3 of the proximal head 2. A longitudinal channel 8 is arranged axially from one end to the other of the screw 1 and opens out at both its opposite ends, this channel 8 being intended to receive a guide pin (not shown). Two self-cutting recesses 9 are arranged at the distal end of the part 5. In the head 2, the axial channel 8 widens out to delimit a seat 11 whose wall constitutes a hexagonal recess 12 adapted to receive a corresponding screwing instrument (not shown).

The external diameter d1 of the threading 7 of the distal part 5 is slightly greater than the diameter d2 of the root 10 of the threading 3 of the head 2. For this reason, given the height of the threads of the threading 3, the external diameter d3 thereof can be of the order of for example 3.1 mm for a diameter d2 of 2.2 mm and a diameter d1 of 2.6 mm. This external diameter d3 of 3.1 mm is a maximum acceptable for the four lateral metatarsals and is also suitable for the first metatarsal which requires screws of a minimal size.

The threading 3 has a maximum depth sufficient to ensure satisfactory bone attachment without otherwise reaching the wall of the hexagonal recess 12.

The fact that the crest of the distal threading 7 has a diameter d1 scarcely greater than the diameter d2 of the root of the threading 3 prevents this distal threading from damaging the entry to the hole in the bone. This is because, in the opposite case, the proximal threading 3 would interfere with the distal cutting of different pitch, which is obviously undesirable for obtaining satisfactory bone anchoring.

If, for example, the external diameter d1 of the distal threading 7 is 2.6 mm for a diameter d2 of 2.2 mm, this gives a radial width of 0.2 mm of the threading 3 in the wall of the bone at the entrance to the hole. This low value of engagement of the threading 3 substantially prevents damage to the bone. It should also be noted that the external diameter d1 of the distal threading 7 cannot be made too small, otherwise it would not ensure sufficient bone attachment.

It has also been found that the best results concerning recesses with different profiles are obtained with a hexagonal profile of the wall 12, and a distance e between two opposite faces 12a of the hexagonal wall 12 of between about 1.3 mm and 1.7 mm. With such a recess, combined with a cannulated screwdriver with a diameter for example of 0.9 mm, it is observed that the screwdriver can exert on the screw 1 a high torsional moment without “slipping” on the hexagonal wall 12.

Trials have established that the external diameter d1 of the distal threading 7 must be between 2.3 and 2.8 mm approximately, the diameter d2 must be between approximately 2.0 mm and 2.5 mm, while the external diameter d3 of the proximal threading 3 must be between substantially 2.8 mm and 3.5 mm. The length of the screw is between 10 and 22 mm. It covers all morphotypes and largely corresponds to the different thicknesses of the metatarsals.

The following table contains an example of the characteristics of a prior art Barouk screw and an embodiment of the self-compressing and atraumatic screw 1 according to the invention.

| | | | 2.5/3 mm screw |
|---|---|---|---|
| | | Current | according to |
| | | 3 mm screw | the invention |
| SCREW | External | 3.9 | 3.1 |
| HEAD | diameter | | |
| | Thread root | 2.67 | 2.20 |
| | diameter | | |
| | Thread height | 0.6 | 0.45 |
| | Pitch | 1 | 0.95 |
| | Engagement | 6 faces | 6 faces 1.5 mm |
| | | 2 mm | |
| | Inter-thread | 0.39 | 0.31 |
| | spacing | | |
| SCREW | External | 3 | 2.6 |
| BODY | diameter | | |
| | Thread root | 1.77 | 1.5 |
| | diameter | | |
| | Thread height | 0.6 | 0.55 |
| | Pitch | 1.34 | 1.25 |
| | Channel | 1.1 | 0.9 |
| | diameter | | |
| | Inter-thread | 0.59 | 0.51 |
| | spacing | | |
| | Tear-out force | 148 | 109 |
| | Torsional | 0.76 | 0.53 |
| | moment | | |

It will be seen that all the dimensions of the screw according to the invention are substantially reduced. Despite this reduction in dimensions, the tear-out force and the torsional moment of the screw remain far greater than those of a separable or twist-off screw having a head with the same external diameter.

FIGS. 4 and 5 illustrate diagrammatically a treatment of hallux valgus which can be carried out using self-compressing and atraumatic screws 1 according to the invention.

Of the five metatarsals 13 and 14, the first metatarsal 14 is deformed in the medial direction (arrow F3 and position 14a indicated by broken lines), constituting metatarsus varus. The surgical procedure for treating this metatarsus varus consists, in brief, in making a longitudinal cut in the metatarsal bone 14 along a longitudinal plane 15 and transverse planes 16 inclined by angles of 60° to the longitudinal plane 15.

In a subsequent step, the surgeon displaces the lower bone fragment 14b laterally, that is to say toward the metatarsals 13. The surgeon then inserts fixation screws 1 in order to firmly close and compress the two bone fragments 14b and 14c on one another. Finally, the surgeon partially cuts the bone part 17 which forms a medial protrusion relative to the bone fragment 14b.

FIG. 6 illustrates a second example of the use of the screw 1 according to the invention, in the context of a Weil osteotomy. In this application, the screw 1 is used to fix the plantar part 13b of a metatarsal 13 which has first been cut longitudinally into two parts 13a and 13b and brought to the desired position.

The pitch of the screws 1 is of the order of 1 mm, substantially equal to that of the larger Barouk screws of the prior art. This increases the hold of the screw in the bone, which is often porotic or of small size. The screw can be made of any suitable biocompatible material, such as a titanium alloy.

1. Self-compressing and atraumatic osteosynthesis screw (1), comprising a threaded proximal head (2), a body (4) forming a threaded distal part (5) and, between the head and the distal part, a smooth section (6) whose length is short compared to the length of the distal part, the threading (7) of the latter having a pitch greater than that of the threading (3) of the proximal head, and a longitudinal channel (8) arranged from one end to, the other of said screw in order to receive a guide pin, characterized in that it includes means allowing it to fix the small bones or small bone fragments of the skeleton after an osteotomy or a fracture, and in particular any one of the five metatarsals (13, 14) of a human foot.

2. Screw according to claim 1, characterized in that said means comprise a diameter (d1) of the threading (7) of the distal part (5) slightly greater than the diameter (d2) of the root of the threading (3) of the proximal head (2), and the fact that the diameter of the channel (8) is between 0.8 and 1.0 mm approximately.

3. Screw according to claim 1 or 2, characterized in that a recess with a hexagonal wall (12) is formed in the proximal head (2) for a screwing instrument, and the distance (e) between two opposite faces (12a) of this recess is approximately 1.3 mm to 1.7 mm.

4. Screw according to one of claims 1 to 3, characterized in that the external diameter (d1) of the threading (7) of the distal part (5) is between 2.3 and 2.8 mm approximately, the diameter (d2) of the root of the threading (3) of the proximal head (2) is approximately 2.0 mm to 2.5 mm, and the external diameter (d3) of the threading of the head is substantially between 2.8 mm and 3.5 mm.
